(19) United States
(12) Patent Application Publication
(10) Pub. No.: US 2010/0316579 A1
(43) Pub. Date: Dec. 16, 2010

(54) NOVEL USE OF ALKYL PHOSPHATE ESTERS

(75) Inventors: Christabel Fowler, Surrey (GB); Gareth David Rees, Surrey (GB)

Correspondence Address: GlaxoSmithKline;GLOBAL PATENTS -US, UW2220, P. O. BOX 1539, KING OF PRUSSIA, PA, 19406-0939, US

(73) Assignee: Glaxo Group Limited

(21) Appl. No.: 12/446,086

(22) PCT Filed: Oct. 25, 2007
(86) PCT No.: PCT/EP2007/061461

§ 371 Date: Apr. 17, 2009

(30) Foreign Application Priority Data
Oct. 26, 2006 (GB) 0621329.2

(51) Int. Cl. A61K 8/55; C07F 9/141; A61K 8/69; A61Q 11/00

(52) U.S. Cl. 424/52; 558/186; 424/57

(57) ABSTRACT

The use of an oral care composition comprising certain alkyl phosphates is described for combating dental erosion and tooth wear.

### FIELD OF THE INVENTION

The present invention relates to the use of an oral care composition comprising certain alkyl phosphates, optionally with a source of fluoride ions, for combating (ie helping to prevent, inhibit and/or treat) dental erosion and/or tooth wear.

### BACKGROUND OF THE INVENTION

Tooth mineral is composed predominantly of calcium hydroxyapatite, Ca₁₀(PO₄)₆(OH)₂, which may be partially substituted with anions such as carbonate or fluoride, and cations such as zinc or magnesium. Tooth mineral may also contain non-apatitic mineral phases such as octacalcium phosphate and calcium carbonate.

Tooth loss may occur as a result of dental caries, which is a multifactorial disease where bacterial acids such as lactic acid produce sub-surface demineralisation that does not fully remineralise, resulting in progressive tissue loss and eventually cavity formation. The presence of a plaque biofilm is a prerequisite for dental caries, and acidogenic bacteria such as Streptococcus mutans may become pathogenic when levels of easily fermentable carbohydrate, such as sucrose, are elevated for extended periods of time.

Even in the absence of disease, loss of dental hard tissues can occur as a result of acid erosion and/or physical tooth wear; these processes are believed to act synergistically. Exposure of the dental hard tissues to acid causes demineralisation, resulting in surface softening and a decrease in mineral density. Under normal physiological conditions, demineralised tissues self-repair through the remineralising effects of saliva. Saliva is supersaturated with respect to calcium and phosphate, and in healthy individuals saliva secretion serves to wash out the acid challenge, and raises the pH so as to alter the equilibrium in favour of mineral deposition.

Dental erosion (i.e. acid erosion or acid wear) is a surface phenomenon that involves demineralisation, and ultimately complete dissolution of the tooth surface by acids that are not of bacterial origin. Most commonly the acid will be of dietary origin, such as citric acid from fruit or carbonated drinks, phosphoric acid from cola drinks and acetic acid such as from vinaigrette. Dental erosion may also be caused by repeated contact with hydrochloric acid (HCl) produced by the stomach, which may enter the oral cavity through an involuntary response such as gastroesophageal reflux, or through an induced response as may be encountered in sufferers of bulimia.

Tooth wear (i.e. physical tooth wear) is caused by attrition and/or abrasion. Attrition occurs when tooth surfaces rub against each other, a form of two-body wear. An often dramatic example is that observed in subjects with bruxism, a grinding habit where the applied forces are high, and is characterised by accelerated wear, particularly on the occlusal surfaces. Abrasion typically occurs as a result of three-body wear and the most common example is that associated with brushing with a toothpaste. In the case of fully mineralised enamel, levels of wear caused by commercially available toothpastes are minimal and of little or no clinical consequence. However, if enamel has been demineralised and softened by exposure to an erosive challenge, the enamel becomes more susceptible to tooth wear. Dentine is much softer than enamel and consequently is more susceptible to wear. Subjects with exposed dentine should avoid the use of highly abrasive toothpastes, such as those based on alumina. Again, softening of dentine by an erosive challenge will increase susceptibility of the tissue to wear.

Dentine is a vital tissue that in vivo is normally covered by enamel or cementum depending on the location i.e. crown versus root respectively. Dentine has a much higher organic content than enamel and its structure is characterised by the presence of fluid-filled tubules that run from the surface of the dentine-enamel or dentine-cementum junction to the odontoblast/pulp interface. It is widely accepted that the origins of dentine hypersensitivity relate to changes in fluid flow in exposed tubules, (the hydrodynamic theory), that result in stimulation of mechanoreceptors thought to be located close to the odontoblast/pulp interface. Not all exposed dentine is sensitive since it is generally covered with a smear layer; an occlusive mixture comprised predominantly of mineral and proteins derived from dentine itself, but also containing organic components from saliva. Over time, the lumen of the tubule may become progressively occluded with mineralised tissue. The formation of reparative dentine in response to trauma or chemical irritation of the pulp is also well documented. Nonetheless, an erosive challenge can remove the smear layer and tubule “plugs” causing outward dentinal fluid flow, making the dentine much more susceptible to external stimuli such as hot, cold and pressure. As previously indicated, an erosive challenge can also make the dentine surface much more susceptible to wear. In addition, dentine hypersensitivity worsens as the diameter of the exposed tubules increases, and since the tubule diameter increases as one proceeds in the direction of the odontoblast/pulp interface, progressive dentine wear can result in an increase in hypersensitivity, especially in cases where dentine wear is rapid.

Loss of the protective enamel layer through erosion and/or acid-mediated wear will expose the underlying dentine, and are therefore primary aetiological factors in the development of dentine hypersensitivity.

It has been claimed that an increased intake of dietary acids, and a move away from formalised meal times, has been accompanied by a rise in the incidence of dental erosion and tooth wear. In view of this, oral care compositions which can help prevent dental erosion and tooth wear would be advantageous.

JP 5-320032 (Kao Corporation) describes a composition for oral use containing an alkyl phosphoric acid ester, a calcium sequestering agent and a phenol derivative. The composition is suggested to have antiplaque activity and anti-acid properties for use in preventing dental caries and periodontal disease. Example 2 of JP 5-320032 presents data for the change in the hardness of enamel challenged with lactic acid when exposed to various compounds and mixtures. The reported data suggests that a combination of an alkyl phosphoric acid ester, a calcium sequestering agent (such as an aluminosilicate zeolite, sodium pyrophosphate or sodium tripolyphosphate) and a phenol (such as ethyl p-hydroxybenzoate, eugenol, thymol, butyl p-hydroxybenzoate or carvacrol) is effective at reducing enamel softening in a caries model based upon a lactic acid challenge. By contrast the data reported in Example 2 suggest that a monoalkyl phosphoric acid ester or a dialkyl phosphoric acid ester is not effective in the absence of a calcium sequestering agent and phenol. Furthermore there is no suggestion of any utility in protecting against dental erosion.

WO 04/075774 (Rhodia) describes compositions containing a surfactant agent consisting essentially of water soluble salts of monoalkyl and dialkyl phosphate esters with a molar ratio of monoesters to diesters of greater than 1. It is suggested that these compounds provide an ablatable coating for anti-adherence of stain and bacteria to teeth; desensitisation of teeth having dental hypersensitivity; low irritancy and improved tissue compatibility or tolerance; increased deposition of various ingredients including anti-microbials and flavour oils; compatibility with peroxide whitening agents, and anti-tartar characteristics. There is no suggestion of any utility in protecting against dental erosion.

Surprisingly it has now been found that demineralisation of dental hard tissues by dietary acids and consequent erosion and/or tooth wear may be reduced or prevented by the use of an oral care composition containing certain alkyl phosphates.

### SUMMARY OF THE INVENTION

Accordingly, the present invention provides the use of an alkyl phosphate of formula (I):

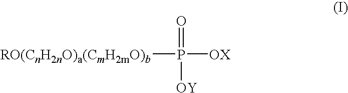

in the manufacture of an oral care composition for combating dental erosion and/or tooth wear, wherein:
R is a C₆-C₂₂ alkyl or alkenyl group,
a and b are individually and separately 0 to 20,
n and m are individually and separately 2 to 4,
X is a counter ion or (CₙH₂ₙO)ₐ(CₘH₂ₘO)_{b}OR as hereinbefore defined, and
Y is hydrogen or a counter ion.

### DETAILED DESCRIPTION OF THE INVENTION

In the alkyl phosphate of formula (I) the alkyl groups may be branched or linear.

R is suitably C₈-C₁₆ alkyl or alkenyl, typically C₁₀-C₁₄ alkyl or alkenyl, for example R is C₁₂ alkyl.

Suitably a and b are individually and separately 0 to 10, for example 0 to 5.

Suitably a and/or b are 0.

When a is greater than or equal to 1, suitably n is 2. When b is greater than or equal to 1, suitably m is 3.

Suitably X is a counter ion.

A counter ion for X or Y is that which forms an orally acceptable salt with an alkyl phosphate. Examples include an alkali metal, an ammonium ion, a protonated alkyl amine, a protonated alkanolamine and a protonated basic amino acid.

Suitable counter ions for X or Y include an alkali metal such as sodium or potassium or an ammonium ion.

Alkyl phosphates for use in the invention include sodium dodecyl phosphate (SDP), potassium dodecyl phosphate (PDP), potassium dodecyl ether (1EO) phosphate (PDEP), sodium 2-ethylhexyl phosphate, sodium di-(2-ethylhexyl)phosphate, sodium di-(dodecyl)phosphate, Tryfac 5559 (CH₃—(CH₂)₁₁₋₁₄—O—(CH₂CH₂O)₅—PO₃K₂) or Crafol AP261 (CH₃—(CH₂)₁₁₋₁₄—O—(CH₂CH₂O)₉—PO₃Na₂) or a mixture of two or more thereof. Many of these alkyl phosphates are available from Rhodia or Cognis.

Whilst the compositions for use in the present invention can include a mixture of a monoalkyl phosphate (where X is a counter ion) with a dialkyl phosphate (where X is (CₙH₂ₙO)ₐ(CₘH₂ₘO)_{b}OR), suitably they contain solely or predominantly a monoalkyl phosphate.

A suitable alkyl phosphate is sodium dodecyl phosphate.

Compounds of formula (I), and mixtures thereof, are known from WO 04/075774 and can be prepared by methods disclosed therein.

Compositions for use in the present invention comprise from 0.01 to 90.0% w/w of alkyl phosphate, suitably from 0.1 to 10.0% w/w, typically from 0.2 to 5.0% w/w, for example from 0.5 to 2.0% w/w.

Suitably the compositions for use in the present invention do not contain a calcium sequestering agent (eg an aluminosilicate zeolite or a chelating agent selected from pyrophosphoric acid, tripolyphosphoric acid, tetrapolyphosphoric acid, citric acid, phytic acid, or EDTA (ethylenediaminetetraacetic acid) or a sodium salt thereof) in combination with a phenol derivative of the type described in the above-noted Kao Corp patent application.

Compositions for use in the present invention may further comprise a source of soluble fluoride ions such as those provided by an alkali metal fluoride such as sodium fluoride, an alkali metal monofluorophosphate such a sodium monofluorophosphate, stannous fluoride, or an amine fluoride in an amount to provide from 25 to 3500 pm of fluoride ions, typically from 50 to 3000 ppm, for example from 100 to 1500 ppm. A suitable source of fluoride ions is an alkali metal fluoride such as sodium fluoride. For example the composition may contain 0.1 to 0.5% by weight of sodium fluoride, eg 0.205% by weight (equating to 927 ppm of fluoride ions), 0.2542% by weight (equating to 1150 ppm of fluoride ions) or 0.315% by weight (equating to 1426 ppm of fluoride ions).

The combination of the alkyl phosphate of formula (I) with a source of fluoride ions provides improved protection against acid demineralisation, dental erosion and/or tooth wear, as evidenced by the data in Examples 1 and 2.

Fluoride ions enhance remineralisation and decrease demineralisation of dental enamel. Therefore the combination of the alkyl phosphate of formula (I) with a source of fluoride ions is of benefit in combating caries in addition to dental erosion.

Compositions for use in the present invention will contain appropriate formulating agents such as abrasives, surfactants, thickening agents, humectants, flavouring agents, sweetening agents, opacifying or colouring agents, preservatives and water, selected from those conventionally used in the oral care composition art for such purposes. Examples of such agents are as described in EP 929287.

Compositions for use in the present invention are typically formulated in the form of toothpastes, sprays, mouthwashes, gels, lozenges, chewing gums, tablets, pastilles, instant powders, oral strips and buccal patches.

Additional oral care actives may be included in the compositions for use in the present invention.

In order to treat dentine hypersensitivity the oral compositions for use in the present invention may further comprise a desensitising amount of a desensitising agent. Examples of desensitising agents include tubule blocking agents or nerve desensitising agents and mixtures thereof, for example as described in WO 02/15809. Suitable desensitising agents include a strontium salt such as strontium chloride, strontium acetate or strontium nitrate or a potassium salt such as potassium citrate, potassium chloride, potassium bicarbonate, potassium gluconate and especially potassium nitrate.

The compositions for use in the present invention may be prepared by admixing the ingredients in the appropriate relative amount in any order that is convenient and which aids solubilisation of the active ingredients and if necessary, adjusting the pH to a desired value. The alkyl phosphate of formula (I) may be solubilised by heating and/or sonication during the manufacture of compositions for use in the present invention.

The present invention also provides a method of combating dental erosion and/or tooth wear which comprises applying an effective amount of a composition comprising an alkyl phosphate as hereinbefore described to an individual in need thereof. Additionally, such a composition has benefit in combating dentine hypersensitivity.

The invention is further illustrated by the following Examples.

### Example 1

### Inhibition of Citric Acid-Mediated Enamel Surface Softening Using SDP and PDEP

The first stage of dental erosion and acid wear involves demineralisation of the hard tissue surface and consequent surface softening. The present study employed a Duramin Microhardness Tester to assess the protective effect of SDP and PDEP against an erosive challenge based on citric acid. A Vickers indentor was employed, and a load of 1.961N applied for 20 seconds.

Sound human enamel specimens were polished with 2400 grit abrasive and subsequently immersed in an aqueous solution of the specified treatment at pH 7 for 2 minutes under ambient conditions, with agitation. After rinsing with deionised water, the enamel specimens were exposed to an erosive challenge comprising an aqueous solution of 0.30% w/w citric acid monohydrate, pH 3.6. The extent of acid damage was assessed by monitoring the decrease in enamel surface hardness as a function of acid exposure time. The microhardness value for each specimen at a given time point was based on the mean of 6 indents. Each treatment leg employed 3 enamel specimens, which were randomised according to baseline microhardness. A 300 ppm fluoride ion solution (from NaF) was employed as the positive control; deionised water was employed as the negative control. The protective effects of combination treatment solutions i.e. 0.50% alkyl phosphate plus 300 ppm fluoride, were also investigated.

The results of this study are shown in FIG. 1 and Table 1. These clearly show that all the active treatments conferred statistically significant levels of protection against the erosive challenge compared to the negative control. Moreover, whilst the levels of protection of SDP, PDEP and the fluoride positive control were comparable at 10 and 20 minutes, SDP and PLEP were statistically superior to the fluoride positive control at 30 minutes. The combination treatments were statistically superior to the single-active treatments at all three time points.

TABLE 1

| | 300 ppm | | | SDP + | PDEP + | |
|---|---|---|---|---|---|---|
| | Fluoride | SDP | PDEP | 300 ppm F | 300 ppm F | Water |
| Baseline | 100 | 100 | 100 | 100 | 100 | 100 |
| 10 mins | 95 ± 1.2 | 94 ± 1.7 | 92.5 ± 1.8 | 98.5 ± 0.8 | 97 ± 1.8 | 73 ± 4.0 |
| Acid | | | | | | |
| 20 mins | 86 ± 2.3 | 84 ± 4.0 | 82.5 ± 2.7 | 95 ± 2.3 | 94 ± 2.1 | 64 ± 2.5 |
| Acid | | | | | | |
| 30 mins | 70 ± 1.5 | 80 ± 2.0 | 78 ± 2.8 | 85 ± 2.6 | 83.5 ± 1.8 | 52 ± 3.1 |
| Acid | | | | | | |

± = Standard Deviation

### Example 2

### Inhibition of Citric Acid-Mediated Enamel Surface Softening by Tryfac 5559 and Crafol AP261

The microindentation protocol described in Example 1 was used to evaluate a number of alkyl polyoxyethylene phosphates including Tryfac 5559 and Crafol AP261. The actives were tested as aqueous solutions at 0.50% w/w and pH 7. The results of this study are shown in FIG. 2 and Table 2. These show that Tryfac 5559, Crafol AP261 and the fluoride positive control give similar and statistically significant inhibition of surface softening at the 20 and 30 minute time points relative to the water control. Of the two alkyl phosphates, Tryfac 5559 appeared to give somewhat greater protection against the citric acid challenge. When Tryfac 5559 was tested in combination with 300 ppm fluoride, no statistically significant improvements were seen compared to the single active treatments, however the combination treatment was directionally superior at 30 minutes.

TABLE 2

| | 300 ppm | | Tryfac + | | |
|---|---|---|---|---|---|
| | Fluoride | Tryfac | 300 ppm F | Crafol | Water |
| Baseline | 100 | 100 | 100 | 100 | 100 |
| 10 mins | 90 ± 1.2 | 84 ± 5.7 | 80 ± 6.0 | 82 ± 2.1 | 78 ± 6.4 |
| Acid | | | | | |
| 20 mins | 81 ± 4.0 | 80 ± 3.5 | 82 ± 6.8 | 75 ± 3.6 | 63 ± 4.5 |
| Acid | | | | | |
| 30 mins | 74 ± 3.8 | 74 ± 1.2 | 79 ± 4.6 | 69 ± 4.6 | 60 ± 1.7 |
| Acid | | | | | |

± = Standard Deviation

### Example 3

### Enamel Solubility Reduction by Alkyl Phosphates Using Citric Acid

FDA caries monograph enamel solubility reduction (ESR) model #33 is designed to evaluate in vitro the utility of fluoride toothpastes to protect enamel against a bacterial (lactic) acid challenge. In brief, enamel specimens are placed in a lactic acid challenge (pH 4.5) and the solubility determined by spectrophotometric analysis of released phosphate. Specimens are then placed in the relevant treatment solution derived from the supernatant of a 1:3 slurry of the toothpaste in deionised water. After 5 minutes the specimens are removed, rinsed, and placed in a fresh lactic acid challenge. The enamel solubility is determined once again, and the ESR value calculated as a percentage reduction relative to the baseline solubility.

The methodology described above was modified in order to evaluate the ability of putative anti-erosion actives to confer protection against a more aggressive dietary acid challenge. In this model variant the lactic acid was replaced with 1.0% w/w citric acid monohydrate pH 3.75. The alkyl phosphates were tested as 0.50% w/w aqueous solutions at pH 7. Fluoride was included as a positive control, and Crest Cavity Protection was also run as an additional control standard. The performance of SDP, PDEP and fluoride is shown in FIG. 3 and Table 3; the data have been normalised with respect to the water negative control.

TABLE 3

| | Enamel Solubility | Standard |
|---|---|---|
| Treatment | Reduction (%) | Deviation |
| PDEP | 17.77 | 4.67 |
| SDP | 36.76 | 3.27 |
| Water | 0.00 | 4.98 |
| 300 ppm Fluoride | 36.16 | 2.02 |
| Crest Regular | 23.43 | 2.81 |

All the active treatments conferred statistically significant protection against the citric acid challenge when compared to the water negative control. SDP was not statistically different to the 300 ppm fluoride control, however PDEP was statistically inferior to both SDP and the fluoride control. SDP was directionally superior to fluoride.

### Example 4

### Enamel Solubility Reduction by a SDP Using Phosphoric Acid

FDA ESR Model #33 was modified by replacing lactic acid with phosphoric acid, a dietary acid most commonly associated with cola drinks. The performance of SDP and fluoride in this phosphoric acid-based ESR model is shown in FIG. 4 and Table 4. Determination of enamel solubility in this study was based on analysis of the released calcium to prevent interference from the acid challenge.

The only treatments that conferred statistically significant protection against the phosphoric acid challenge when compared to the negative water control were SDP and the 300 ppm fluoride. Treatment with SDP conferred statistically superior acid protection versus the 300 ppm fluoride.

TABLE 4

| | Enamel Solubility | Standard |
|---|---|---|
| Treatment | Reduction (%) | Deviation |
| Water | 0.00 | 2.56 |
| Crest Regular | 1.22 | 2.53 |
| SDP | 16.02 | 3.07 |
| 300 ppm Fluoride | 6.29 | 2.36 |

1. The use of an alkyl phosphate of formula (I): 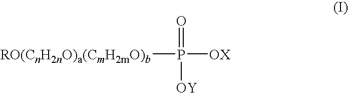
in the manufacture of an oral care composition for combating dental erosion and/or tooth wear, wherein:
R is a C₆-C₂₂ alkyl or alkenyl group,
a and b are individually and separately 0 to 20,
n and m are individually and separately 2 to 4,
X is a counter ion or (CₙH₂ₙO)ₐ(CₘH₂ₘO)_{b}OR as hereinbefore defined, and
Y is hydrogen or a counter ion.

2. The use according to claim 1 wherein R is C₁₀-C₁₄ alkyl or alkenyl.

3. The use according to claim 1 wherein R is C₁₂ alkyl.

4. The use according to claim 1 wherein a and/or b are 0.

5. The use according to claim 1 wherein X is a counter ion.

6. The use according to claim 1 wherein a counter ion is an alkali metal or an ammonium ion.

7. The use according to claim 1 wherein the alkyl phosphate is selected from sodium dodecyl phosphate (SDP), potassium dodecyl phosphate (PDP), potassium dodecyl ether (1EO) phosphate (PDEP), sodium 2-ethylhexyl phosphate, sodium di-(2-ethylhexyl)phosphate, sodium di-(dodecyl)phosphate, Tryfac 5559 (CH₃—(CH₂)₁₁₋₁₄—O—(CH₂CH₂O)₅—PO₃K₂) and Crafol AP261 (CH₃—(CH₂)₁₁₋₁₄—O—(CH₂CH₂O)₉—PO₃Na₂) and a mixture of two or more thereof.

8. The use according to claim 1 wherein the alkyl phosphate is sodium dodecyl phosphate.

9. The use according to claim 1 wherein the oral composition further comprises a source of fluoride ions.

10. The use according to claim 1 wherein the oral composition further comprises a desensitising agent.

11. A method for combating dental erosion and/or toothwear which comprises applying an effective amount of an oral care composition as claimed in claim 1 to an individual in need thereof.
